(19) United States
(12) Patent Application Publication
(10) Pub. No.: US 2019/0049377 A1
(43) Pub. Date: Feb. 14, 2019

(54) BIOAFFINITY ASSAY METHOD UTILIZING TWO-PHOTONEXCITATION OF FLUORESCENCE

(71) Applicant: Arcdia International Oy Ltd, Turku (FI)

(72) Inventors: Niko PORJO, Kaarina (FI); Jori SOUKKA, Vanhalinna (FI)

(73) Assignee: Arcdia International Oy Ltd, Turku (FI)

(21) Appl. No.: 16/076,682

(22) PCT Filed: Feb. 23, 2017
(86) PCT No.: PCT/FI2017/050117

§ 371 Date: Aug. 8, 2018

(30) Foreign Application Priority Data
Feb. 25, 2016 (FI) 20165148

(51) Int. Cl. G01N 21/64; G01N 33/543

(52) U.S. Cl. CPC G01N 21/6408; G01N 21/6428; G01N 2021/6415; G01N 2021/6439; G01N 33/54313

(57) ABSTRACT

The invention relates to a separation free bioanalytical assay method for qualitatively and/or quantitatively determining an analyte (4) in a sample of a biological fluid or suspension. The invention resides in that the method comprises, apart from essential steps for a two-photon excitation based assay method well known in prior art, the further steps of: a) recording focus positions and corresponding two-photon excited fluorescence emission photon counts of a plurality of microparticles (1) of a device; b) calculating a correction matrix for the device employing the recorded focus positions and corresponding two-photon excited fluorescence emission photon counts, and c) correcting two-photon excited fluorescence emission photon counts from the microparticles (1) of said device employing the correction matrix obtained for the device employing the recorded focus positions and the corresponding two-photon excited fluorescence emission counts.

### TECHNICAL FIELD OF THE INVENTION

The present invention relates to in vitro diagnostic assays and to the use of two-photon excited fluorescence as a detection principle for the measurement of bioaffinity assays.

### BACKGROUND OF THE INVENTION

The publications and other materials used herein to illustrate the background of the invention, and in particular, cases to provide additional details respecting the practice, are incorporated by reference.

Applications of Fluorescence in Bioaffinity Assays

One-photon excited fluorescence has found various applications in the field of bioanalytics. Applications such as immunoassays, DNA-hybridization assays and receptor binding assays using fluorescence as a detection method have been introduced during the last decades. These assays utilize specific bioaffinity reactions in determination of the analyte in a sample. The amount of the analyte can be determined by monitoring the fluorescence signal that depends on the amount of the bound analyte. These assays can also be based on monitoring of the change in the fluorescence properties upon a specific binding reaction. This change in the fluorescence property can be a change in the fluorescence intensity, a change in the emission wavelength, a change in the decay time or in the fluorescence polarization.

Immunoassays have been used extensively in in vitro diagnostics for determination of certain diseases or a physiological condition. Immunoassays can be categorized to two different types of assays, competitive and non-competitive assays. In a competitive method, a labelled antigen (secondary biospecific reagent) competes with the analyte in binding to a limited quantity of antibody (primary biospecific reagent). The concentration of the analyte can be determined from the proportion of the labelled antigen bound to the antibody or from the proportion of the free fraction of the labelled antigen. In a non-competitive method (immunometric method) the analyte is bound to an excess amount of binding antibody (primary biospecific reagent). An excess of the labelled antibody (secondary biospecific reagent) binds to another site of the analyte. The amount of the analyte can be determined on basis of the fraction of the labelled antibody bound to the analyte. Physical separation of the bound and free fractions is normally necessary before the detection unless the detection principle is able to distinguish the signal of the bound fraction from the signal of the free fraction. Thus, the assay methods are divided in to separation assays and separation-free assays, often also called as heterogeneous and homogeneous assays. [Miyai K., Principles and Practice of Immunoassay, (ed. Price C. P. and Newman D. J.) Stockton Press, New York 1991, 246 and Hemmilä I. A., Applications of Fluorescence in Immunoassays, (ed. Winefordner J. D.) John Wiley & Sons, New York 1991].

Two-Photon Excited Fluorescence

Two-photon excitation is created when, by focusing an intensive light source, the density of photons per unit volume and per unit time becomes high enough for two photons to be simultaneously absorbed by the same chromophore. The absorbed energy is the sum of the energies of the two photons. The probability of two-photon excitation is dependent on the 2nd power of the photon density. The absorption of two photons is thus a non-linear process of the second order. The simultaneous absorption of the two photons by one chromophore yields a chromophore in excited state. This excited state is then relaxed by spontaneous emission of a photon with higher energy than the photons of the illumination. In this context the process that includes two-photon excitation and subsequent radiative relaxation is called two-photon excited fluorescence. TPE has usually similar emission properties to those of one-photon excited fluorescence of the same chromophore [Xu C. and Webb W. W., J. Opt. Soc. Am. B, 13 (1996) 481].

One of the key features of two-photon excitation is that excitation takes place only in a clearly restricted 3-dimensional (3D) vicinity of the focal point. The outcome of this feature is high 3D spatial concentration of the generated fluorescence emission. Due to the non-linear nature of excitation, minimal background fluorescence is generated outside the focal volume, i.e. in the surrounding sample medium and in the optical components. Another key feature of two-photon excitation is that illumination and emission takes place in essentially different wavelength ranges. A consequence of this property is that leakage of scattered illumination light in the detection channel of the fluorescence emission can be easily attenuated by using low-pass filters (attenuation of at least 10 orders of magnitude). Since the excitation volume is very small (in the range of femtoliters, i.e. 10⁻¹⁵ liters), two-photon excitation is most suitable for observation of small sample volumes and structures.

If the cuvette was covered with a foil (or other type of cover) and the dispensing of the samples is carried out through the foil with a thin dispensing needle, probability for spilling would be decreased when compared to open cuvettes. In such a case, the probability of spilling would be proportional to the diameter of the piercing needle. However, even in this case, spilling is very likely to occur during shaking and significant evaporation is likely to occur during incubation. These can deteriorate assay performance.

Bioanalytical Applications Utilizing Two-Photon Excited Fluorescence

One of the early reports relative to analytical applications of two-photon excitation was published by Sepaniak et al. [Anal. Chem. 49 (1977), 1554]. They discussed the possibility of using two-photon fluorescence excitation for HPLC detection. Low background and simplicity of the system were demonstrated. Lakowicz et al. [J. Biomolec. Screening 4 (1999) 355] have reported the use of multi-photon excitation in high throughput screening applications. They have shown that two-photon-induced fluorescence of fluorescein can be reliably measured in high-density multi-well plates.

Most of the bioanalytical applications of two-photon excited fluorescence that are described in the literature relate to two-photon imaging microscopy [Denk W. et al. U.S. 5,034,613, Denk W. et al., Science 248 (1990) 73]. The use of two-photon fluorescence excitation in laser scanning microscopy provides inherent 3D spatial resolution without the use of pinholes, a necessity in confocal microscopy. With a simple optical design two-photon excitation microscopy provides comparable 3D spatial resolution to that of ordinary one-photon excited confocal microscopy. The development has also lead to industrial manufacture of two-photon laser scanning microscope systems. The disadvantage of the two-photon excitation technology is the need of an expensive laser capable of generating intense ultra-short pulses with a high repetition frequency.

The development of less expensive laser technology has enabled the use of two-photon fluorescence excitation technology in routine bioanalytical applications [Hänninen P. et al., Nat. Biotechnol. 18 (2000) 548; Soini J. T. et al. Single Mol. 1 (2000) 203; Soini J T (2002) Crit. Rev. Sci. Instr., WO 98/25143, WO 99/63344 and WO 05/078438]. According to WO 98/25143, WO 99/63344, and WO 05/078438 instead of expensive mode-locked lasers, passively Q-switched diode-pumped microchip lasers can be used for two-photon excitation. These lasers are monolithic, small, simple and low in cost. WO 98/25143 and WO 99/63344 describe the use of two-photon excited fluorescence in detection bioaffinity assay. This bioaffinity assay technique employs microparticles as a bioaffinity binding solid phase to which a primary biospecific reagent is bound. This bioaffinity assay technique utilizes a biospecific secondary reagent that is labelled with a two-photon fluorescent dye. According to the methods described in WO 98/25143 and WO 99/63344, bioaffinity complexes are formed on the surface of microparticles, and the amount of bioaffinity complexes is quantified by measuring two-photon excited fluorescence from individual microparticles. Thus, this assay technique enables separation-free bioaffinity assays in microvolumes.

The labelled secondary bioaffinity reagent binds on the surface of microparticles either via an analyte molecule to form three component bioaffinity complexes (non-competitive, immunometric method) or it binds directly to the primary biospecific reagent to form two component bioaffinity complexes (competitive binding method). The primary and secondary biospecific reagents are biologically active molecules, such as haptens, biologically active ligands, drugs, peptides, polypeptides, proteins, antibodies, or fragments of antibodies, nucleotides, oligonucleotides or nucleic acids. According to WO 98/25143 and WO 99/63344 a laser with high two-photon excitation efficiency is focused into the reaction suspension and two-photon excited fluorescence is measured from single microparticles when they float through the focal volume of the laser beam. Alternatively the microparticles can be trapped for a period of fluorescence detection with an optical trap, which is brought about with a laser beam. The trapping of microparticles to the focal point of the laser beam is based on optical pressure that is generated onto the microparticle by the illuminating laser. Microparticles are actively searched from the reaction suspension by a two dimensional pietzo driven scanner. The scanner is capable to stop the scan action momentarily when a microparticle is found in the vicinity of the focal volume. The fluorescence signal from individual microparticles is detected by a photomultiplier tube.

### OBJECT AND SUMMARY OF THE INVENTION

The object of the present invention is to provide an improved separation free bioanalytical assay method for qualitatively and/or quantitatively determining an analyte in a sample of a biological fluid or suspension, said method comprising the steps of:

a) contacting a bioaffinity solid phase comprising microparticles to which a primary reagent biospecific to said analyte is bound simultaneously with said sample and a secondary reagent biospecific to said analyte labelled with a fluorescent label in a reaction volume, thereby initiating a reaction,

b) scanning a two-photon excitation focal volume within said reaction volume using a beam deflecting scanner and a two-photon exciting volume created by a laser beam which optically moves the microparticles,

c) momentarily interrupting scanning or reducing scanning speed of said two-photon excitation focal volume when said two-photon exciting volume approaches a microparticle randomly located in the reaction volume,

d) applying optical force to said microparticle such that it moves into and in the two-photon exciting volume created by said laser beam, and

e) detecting two-photon excited fluorescence emission photon counts from said microparticle.

Thus the present invention provides such a separation free bioanalytical assay method for qualitatively and/or quantitatively determining an analyte in a sample of a biological fluid or suspension, the method further comprising the steps of:

f) recording focus positions and corresponding two-photon excited fluorescence emission photon counts of a plurality of said microparticles of a device;

g) calculating a correction matrix for said device by employing said recorded focus positions and said corresponding two-photon excited fluorescence emission photon counts, and

h) correcting two-photon excited fluorescence emission photon counts from said microparticles of said device by employing said correction matrix obtained for said device by employing said recorded focus positions and said corresponding two-photon excited fluorescence emission counts.

### BRIEF DESCRIPTION OF THE DRAWINGS

FIG. 1 schematically shows reaction mixture constituents, solid phase reaction carriers, free fluorescent antibody tracer, analyte, formed three component immunocomplexes and non-binding fluorescent substances from sample matrix.

FIG. 2 schematically shows a cross-section of an optical arrangement for scanning a solution with focused light.

FIG. 3 schematically shows a particle acceptance volume, variable illumination intensity and sensitivity.

FIG. 4 schematically shows an optical arrangement.

FIG. 5 schematically shows a preferred implementation of an adaptive correction method according to the invention.

FIG. 6 schematically shows a detail on the implementation of an adaptive correction method according to the invention.

FIG. 7 shows a histogram of signal values from a selection of particles.

FIG. 8 shows a contour plot of an example correction matrix.

FIG. 9 shows a series of contour plots illustrating the change in the correction matrix over time.

### DETAILED DESCRIPTION OF THE INVENTION

### Technical Problem

When setting cut-off values for a qualitative in vitro diagnostics assay, sensitivity and specificity are typically interchangeable. When the diagnostic analysis is based on the measurement of fluorescence properties of suspended solid phase particles there is an inherent variation in the fluorescence brightness of individual particles, caused by biochemical variation and variations in how the particle enters the focal point, and thus typically several particles need to be measured to more accurately assess the actual signal and hence, the concentration of the target analyte. The mean brightness of the particles of the bioaffinity assay is dependent on the state of the reaction between the reagents and the target analyte, and their concentrations. Any variation in the measurement device will lead to a larger variation in the results and lead to either requiring the measurement of more particles, which require a longer fluorescence scanning time, or a higher cut-off, both of which are undesirable.

Controlling the location of suspended particles adds cost and complexity to a device. Additionally for a two-photon fluorescence excitation system the power density required to achieve fluorescence is so large that it can only be achieved in a small volume at one time and even then only in relatively short pulses. This is due both to the availability of light sources and to the limited ability of the suspension fluid to reject excess heat. For these reasons a system of scanners is used to find particles in the suspension. In this type of scanning system the particles are found in effectively random locations within the scanned volume.

The analyte may be but is not limited to the group consisting of a hapten, biologically active ligand, drug, peptide, oligonucleotide, nucleotide, nucleic acid, polypeptide, protein, antibody, a fragment of antibody, a carbohydrate, a micro-organism, a cell or a group of cells. The size of the solid particle can also vary over several orders of magnitude at least from hundreds of nanometers to tens of micrometers, though generally in one application, one type of particle is used at a time. The concentration of the analytes can also vary from only a few individual analytes per reaction to very large quantities. This means that even for the same measurement device using the same particles the measured fluorescence intensity can be non-linearly dependent on the concentration and may depend on the analyte.

Prior art shows that illumination intensity and detection sensitivity differences in the field of view of an optical apparatus are common problems and several solutions have been devised to correct them. For a system using non-linear excitation this problem can be particularly severe or in other words the measured fluorescence intensity is more sensitive to changes in illumination intensity compared to for example bright field fluorescence excitation. The problem is exacerbated when system throughput issues push to measure as few particles as possible. When the number of particles is low the probability of finding all the particles in areas of high or low illumination intensity and high or low fluorescence reading sensitivity starts to dominate the setting of the cut-off, i.e. the cut-off must be raised or otherwise the test specificity suffers.

Factors that contribute to the variation of illumination intensity and sensitivity between locations in the measurement volume include optical aberrations in the objective and other optical components, manufacturing tolerances in the mechanical construction and assembly tolerances. While many of these can be reduced it can lead to expensive and bulky designs. Further, many of these factors may change over the lifetime of the device. Objective performance may change due to dust and other impurities on surfaces, vibration and impact shocks may change the tuning of the optical path.

Additional time dependent variance sources are laser power which changes due to ageing and changes in adhesives that are used to attach mirrors to their holders.

The three dimensional shape and size of the focus volume where the probability of two-photon excitation is high will change due to the above mentioned time dependent changes in the device. These changes will couple to the measurement as the optical forces that affect the trajectory of the particle during the measurement will change. The time spent in the focus will change as well as the position relative to the high intensity part of the focus volume. These will in turn change the ratio between the fluorescence obtained from surface bound fluorescent tracer and the tracer that is suspended in the solution but which will also be excited by the laser during the particle measurement. Additional effects due to changes in the focus will be seen if optical phenomena such as surface plasmon resonance are used to enhance the fluorescence signal.

All of the above problems will affect how often the device needs maintenance as well as what type of maintenance is needed. Moreover, typically external calibration is needed regularly to compensate for changes from assay to assay and over time. External calibration is often tedious and interferes the use of the device for routine diagnostic testing.

The validity of the results depends on the stability of the device. If there are changes in the device during operation it is important for the device to be able to detect changes that will lead to violation of specifications.

### Solution

Location dependence of the signal is measured using the fluorescence values given by particles measured from clinical samples. Location dependence is measured from the combination of particles from clinical samples and particles designed to have similar susceptibility to fluorescence as particles in the clinical samples that are close to the average brightness that result in measurement result close to the cut-off. When cut-offs for different analytes differ so much that significant differences occur, calculation of the correction matrix is done separately for each analyte.

When a multiplex assay format is used and it is possible to identify the measured analyte from a single particle measurement either directly from the fluorescence or through indirect means such as differences in light scattering or from inherently fluorescent particles a necessary number of different correction matrices may be created to optimally minimize location dependent variation for each of the multiplexed tests.

Recalculation of the correction surface is done whenever suitable particles are measured or a suitable time period has passed. At the beginning of use for a certain device a calibration measurement done at the factory may be used as the particle set that defines the initial correction values. To reduce noise in the correction values to an acceptable level many particles need to be measured and the length of time from which particles are accepted to the calculation may be changed to achieve suitable noise levels and at the same time make the correction matrix reactive to changes in the system.

Changes in the correction values are used to monitor the health of the device. If the values change beyond preset limits the device may report an error and suspend operation pending maintenance or evaluation of the problem. If the rate of change of the correction values exceeds a predetermined value maintenance may be rescheduled or a replacement device may be prepared before the device suspends operation.

### Advantageous of Invention

Because of better real-time monitoring of the device health the probability that the device is out of specification is smaller.

Early detection of problems lowers user downtime and enhances system reliability.

Test sensitivity, specificity, precision, and/or accuracy are improved. This is especially true when the solution background signal is measured separately and subtracted from the measurement signal obtained from the particles, and the sample matrix causes high solution background signal. Employing the invention allows one to improve precision of quantitative analytical analysis. Improved precision allows the use of lower cut-offs while maintaining high specificity as condition negative sample results are within a narrower window. This improves sensitivity and/or specificity and finally allows for better accuracy in qualitative tests.

Terms

Terms used in this application can be defined as follows:
Bioaffinity assays: A common name for all bioassays that are based on bioaffinity binding reactions, i.e. a reaction where bioaffinity complexes are formed.
Correction matrix: In the preferred embodiment a two dimensional rectangular array of numbers arranged in rows and columns. More generally a k-dimensional array with dimension size n1 . . . nk.
Dichroic mirror: Is a mirror which reflects selected bands of electromagnetic radiation and passes others.
Error status: A number or description related to a measurement result in the database. In many cases when one quantity is measured simultaneous measurement of other quantities is done, these may indicate that the measurement result is less accurate than optimal or is the result of interference.
Microparticle: A particle typically close to spherical shape with dimensions in the micrometer scale. The described measurement system can also use smaller or larger particles but typically micrometer scale particles are the best choice.
Q-switched: Or giant pulse formation or Q-spoiling. A technique where the laser resonators Q-value is reduced significantly while the gain medium is pumped and lacing only occurs when the Q-switch releases. Results in very high peak power for a pulsed laser.
Two-photon excitation (TPE): A phenomenon where two photons excite a fluorophore in a single quantum event. Makes it possible to effectively filter the high power exciting light from the low power fluorescence light created by the fluorophores. Due to low two-photon excitation cross-sections very high power densities are required two produce two-photon excitation, these can be achieved by focusing a laser beam. An added benefit is that excitation at the fluorophore working wavelength outside the focal volume becomes very unlikely and thus a separation free assay is possible.
Two-photon excited fluorescence: Light produced by two-photon excitation.

Does not differ from ordinary light except for the origin, used here to draw attention to the wavelength.
Well: A small container used here to hold the reagents and suspension fluid. Typically arranged in strips or arrays in plates. In the preferred embodiment has an optical quality window at the bottom of the well.

Preferred Embodiments of Invention

A typical embodiment of the invention comprises a separation free bioanalytical assay method for qualitatively and/or quantitatively determining an analyte in a sample of a biological fluid or suspension, said method comprising the steps of:

a) contacting a bioaffinity solid phase comprising microparticles to which a primary reagent biospecific to said analyte is bound simultaneously with said sample and a secondary reagent biospecific to said analyte labelled with a fluorescent label in a reaction volume, thereby initiating a reaction,

b) scanning a two-photon excitation focal volume within said reaction volume using a beam deflecting scanner and a two-photon exciting volume created by a focused laser beam which optically moves the microparticles,

c) momentarily interrupting scanning or reducing scanning speed of said two-photon excitation focal volume when said two-photon exciting volume approaches a microparticle randomly located in the reaction volume,

d) applying optical force to said microparticle such that it moves into and in the two-photon exciting volume created by said laser beam, and

e) detecting two-photon excited fluorescence emission photon counts from said microparticle;

characterized in that said method further comprises:

f) recording focus positions and corresponding two-photon excited fluorescence emission photon counts of a plurality of said microparticles of a device;

g) calculating a correction matrix for said device by employing said recorded focus positions and said corresponding two-photon excited fluorescence emission photon counts, and

h) correcting two-photon excited fluorescence emission photon counts from said microparticles of said device by employing said correction matrix obtained for said device by employing said recorded focus positions and said corresponding two-photon excited fluorescence emission counts.

In most typical embodiments of the present invention the correction matrix is recalculated continuously, or at pre-set intervals or time points, by employing the recorded focus positions and the corresponding two-photon excited fluorescence emission photon counts within a defined preceding time period. Preferably the preceding time period is chosen so that recorded focus positions and corresponding two-photon excited fluorescence emission photon counts of a minimum number of microparticles are employed when calculating the correction matrix for the device.

In some preferred embodiments of the invention the correction matrix is calculated by employing the recorded focus positions and the corresponding two-photon excited fluorescence emission photon counts from microparticles of at least one negative control sample, i.e. a sample or samples not comprising the analyte.

In other preferred embodiments of the invention the correction matrix is calculated by employing recorded focus positions and corresponding two-photon excited fluorescence emission photon counts of clinical sample measurements and employing only particles with two-photon excited fluorescence emission photon counts within a predetermined margin of the cut-off value for a positive result for an analyte.

In many preferred embodiments of the invention the two-photon excited fluorescence emission photon counts from individual microparticles are normalized for the median of the fluorescence emission photon counts obtained during the measurement of a single well.

In many embodiments of the invention the correction matrix is approximated by calculating an n by m matrix of correction factors where for each position of the correction matrix an approximate correction value is calculated from the two-photon excited fluorescence emission photon counts from said microparticles that were detected within a set radius from said position. Preferably the approximate correction value is the median of the two-photon excited fluorescence emission photon counts.

In some preferred embodiments of the invention changes in the correction matrix are applied to determine changes in the health of the device, i.e. in device health, and/or need for maintenance of the device.

In some embodiments of the invention the device is withdrawn from use until maintenance if the correction matrix changes beyond a set limit.

In other embodiments of the invention the device is withdrawn from use until maintenance if the speed of change of the correction matrix exceeds a set limit.

When considering the disclosure of this description a person skilled in the art would understand that preferred embodiments of the invention would comprise embodiments with any combination of the features disclosed unless a person skilled in the art would understand that such features would clearly exclude one another.

Particular Embodiments

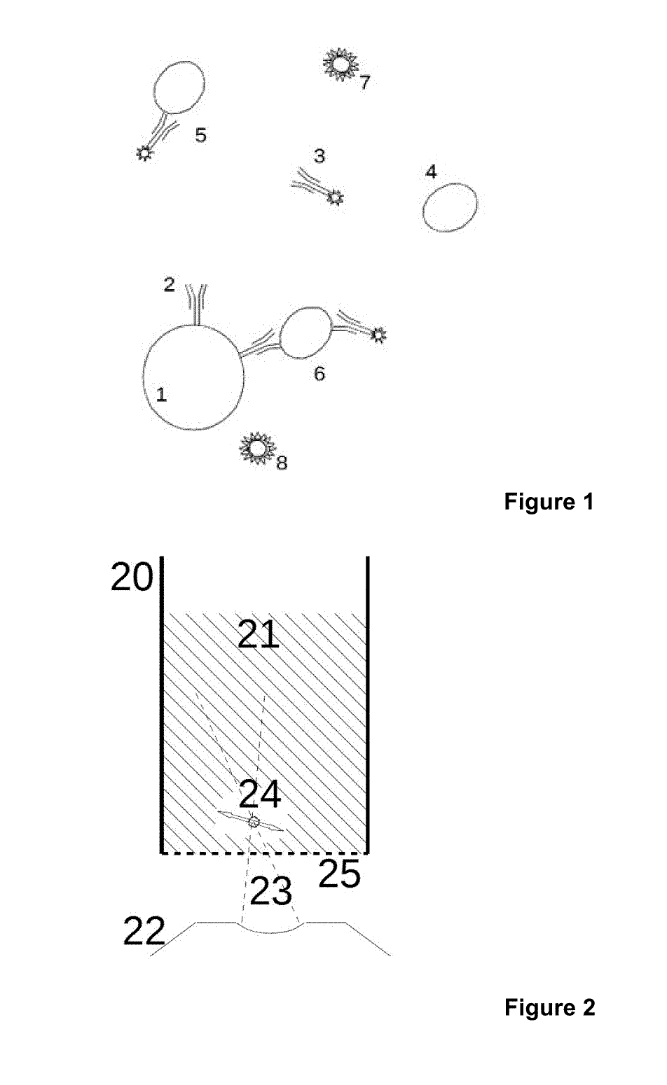

In a preferred embodiment a sample of a biological fluid or suspension containing analytes 4 (see FIG. 1) are mixed to a buffer fluid solution containing microparticles 1 coated with biospecific primary reagent such as antibodies 2 and a two-photon excitable fluorescent tracer, a biospecific secondary reagent labelled with a fluorescent label 3. During the immunocomplex formation analytes 4, the tracer 3 and the biospecific primary reagent 2 become bound and concentrated from the solution onto the microparticles 1. During the measurement, microparticles 1 are sought with the focused laser beam and the two-photon fluorescence from the surface bound tracer is measured.

When the beam is scanned in the solution the amount of back scattered light is constantly measured. If a threshold is exceeded the scanners are stopped and measurement of the particle is started.

The solution is mixed periodically to keep the microparticles in the suspension and to avoid the formation of concentration gradients which would slow the reactions.

Background fluorescence may be emitted in a separation free assay by the free tracer 3 or by other fluorescent molecules 7, 8 brought to the reaction mixture in the sample matrix. FIG. 1 is not drawn to scale and the relative sizes of the participating compounds may differ by orders of magnitude without requiring changes to the measurement apparatus. It is possible that bound tracer 6, other fluorescent molecules 7, 8 and the free tracer 3 or a combination of these are measured at the same time. This is a consequence of the desirable separation free nature of the assay.

The sample solution 21 (see FIG. 2) is moved to a cuvette or a well 20 on a test plate. An objective lens 22 focuses the laser beam to a point 24 in the well 20 through a window 25. Dashed lines 23 show a cross-section of the cone of light produced by the objective lens 22 with the focal point 24 at its waist. The arrow at the focal point represents the ability of the complete system to move the focal point 24 in the well 20.

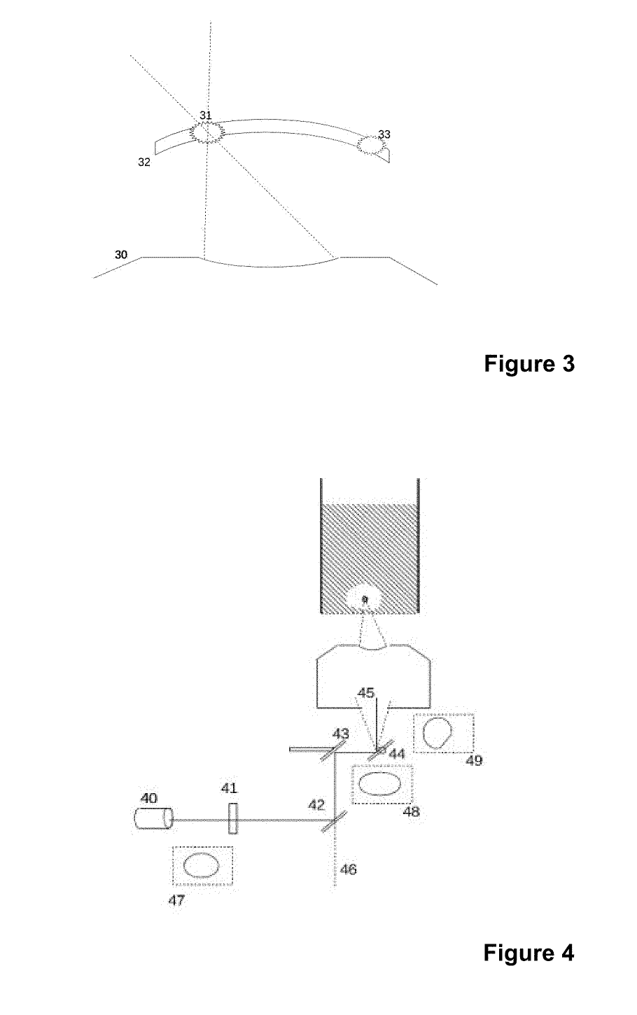

When the focal point 31 (see FIG. 3) scans the sample solution it moves along a surface 32 to various positions such as to a position 33. While the cross-section 32 is curved the actual surface may be a complex shape. Due to the properties of the optical system, the illumination intensity at the focal points 31, 33 can be different. In the preferred embodiment, the light intensity at the focal point is so large that the microparticles are actuated by the electromagnetic fields and move towards the focal point and through it. This is represented by the thickness in the drawing of the surface 32.

FIG. 4 shows a simplification of the optical arrangement to show several sources for the intensity variation at the scanned focal point. A laser 40 creates a beam which passes through a lens 41 and is reflected through a dichroic mirror 42, to a first scanning mirror 43 where it is reflected to a second scanning mirror 44. The beam is then reflected to an objective lens. The direction of the beam depends on the configuration of the mirrors 43 and 44 as shown by the solid and dashed lines 45.

The scanning mirrors 43 and 44 are rotated on a perpendicular axis. The focal length of the lens 41 is used to control the divergence of the beam so that the entrance pupil of the objective lens is correctly filled with the beam.

When leaving the laser the beam typically has a Gaussian intensity profile and an elliptic or quasi circular cross-section 47. After being reflected from several mirrors the cross-section of the beam changes as shown by 48. Because of its Gaussian intensity profile the beam is cut at the edges even in the best case, imperfections may cause more severe and asymmetric cutting as shown in 49. All the profile representations 47, 48 and 49 show the border of constant intensity.

Changing the configuration of the scanning mirrors 43 and 44 then causes the focal point to move in the sample solution. Some of the scattered and two-photon excited fluorescence is collected by the objective and reverses the optical path. The dichroic mirror 42 is selected so that two-photon excited fluorescence passes it 46. Two-photon excited fluorescence is then collected and transduced to an electrical signal by a sensor, typically a photomultiplier tube.

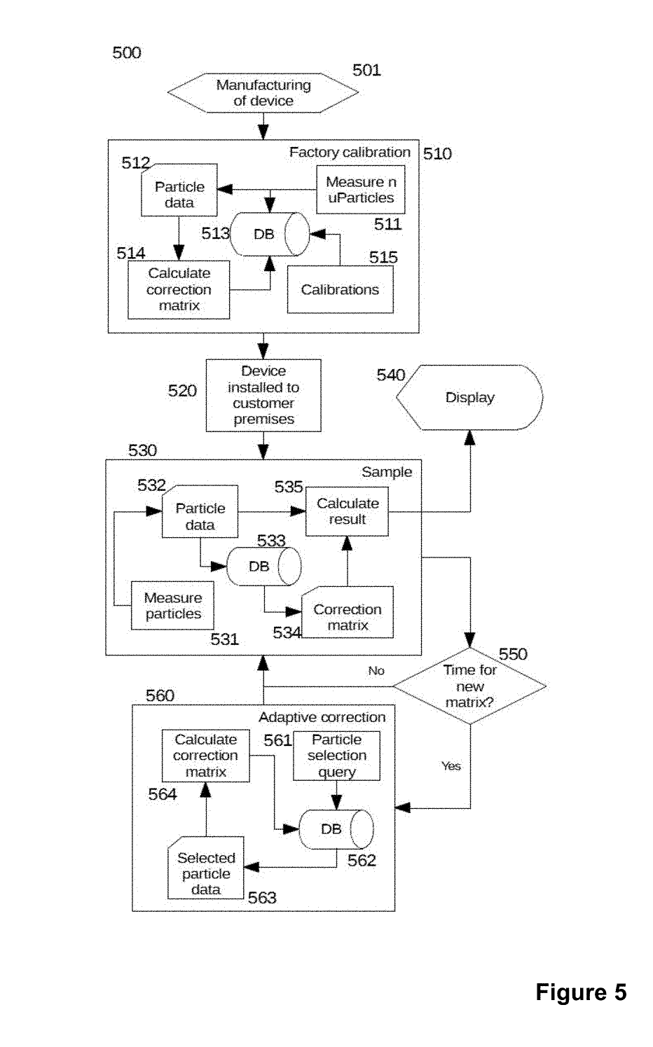

Flowchart 500 in FIG. 5 shows when initial calibrations are done and when the first correction matrices are created. Manufacturing of a device (501) includes both creation of a physical instance and installing of the embedded software. When the measurement device comprises multiple units, some of which may be off the shelf, such as a PC, this phase may also include installing other software components.

Factory calibration (510) may include several different calibration steps as shown by the calibrations process step (515). The adaptive correction of this invention requires the measurement of a number of particles (511). Here n may depend on several factors, including the spatial distribution of the microspheres, as a low density in any area of the scanned surface may lead to excessive noise in a correction matrix. The result is a particle data set (512) which in addition to the fluorescence value for each particle may include other information that can be used for example to add weighting factors to the particles.

Step 514 then creates a matrix that can be used to correct particle values at each point of the scanned area. Depending on the application, for example when several different assays are measured with the same physical device, a multidimensional array may be created. In that case different assays may have differing correction matrices to optimize the calibration, for example when the sandwich participants vary in size between assays. The resulting array is then injected to the database (513) and the factory calibration block ends.

When the device is installed to customer premises (520) additional calibration steps may be performed to ensure that no changes have occurred during the transportation and a possible storage.

In normal use, a sample (530) is inserted to the device and the analyser starts to measure particles (531) and a background signal from the solution. This results in particle data (532) which is again injected to the database (533). In calculate result (535) this data and the correction matrix (534) extracted from the database (533) is used to calculate the corrected result (535). The result is then shown to the user (540) or sent outside the device.

After the sample has been analysed the system checks if it is time to recalculate the correction matrix (550). This check makes it possible to optimize the system performance when the available computational power is limited. If No, the next sample may be measured when available. If Yes, the adaptive correction step (560) is entered. Here a particle selection query (561) is formed and used to extract selected particle data (563) from the database (562). This data is again used to calculate a correction matrix (564), which is injected to the database (562).

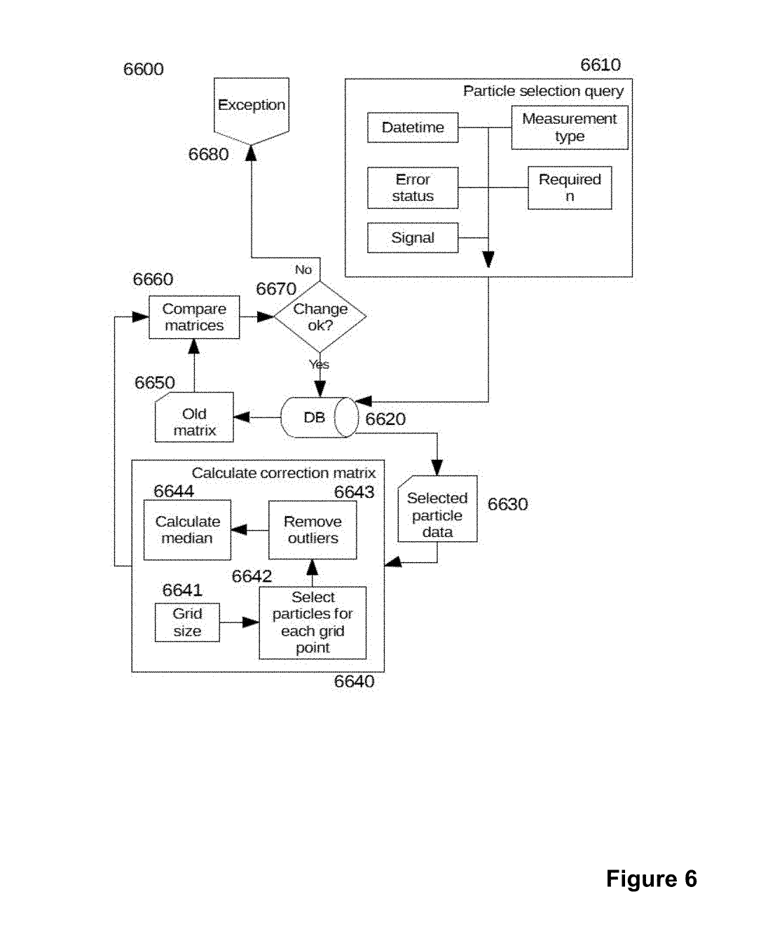

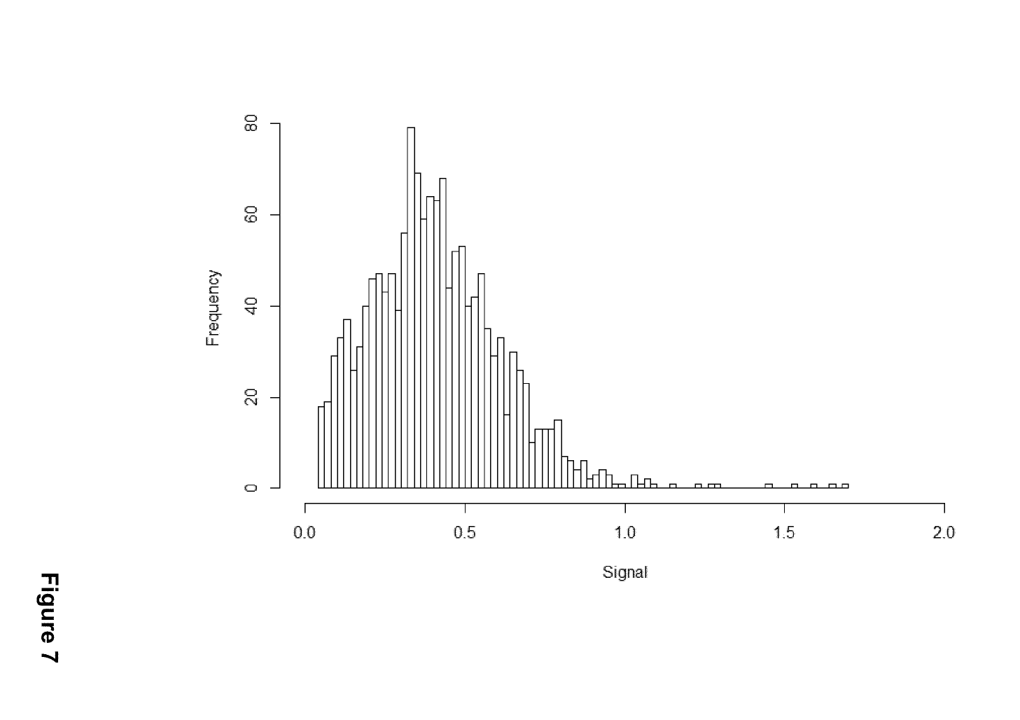

Flowchart 6600 in FIG. 6 shows details of the adaptive correction process. The particle selection query (6610) comprises several sub-processes which collect information required to select the correct particles. Date and time is used to select the samples that have been measured recently, for example only particles measured during the last month might be accepted. This may lead to a situation where the required number of particles cannot be selected, and the calibration may need to be aborted. An alternate and preferred solution is to use a required number of newest particles.

Particles may have an error status attached to them, in some cases the error may not be relevant from the correction point of view and the particle may still be used. Because the correction is most important close to any decision threshold the system may have, such as a cut-off for a qualitative result, it is advantageous to select particles which have a signal value that is within a predetermined range from the said threshold. When the database includes measurements for several different methods (analytes) either from separate tests or multiplexed tests it is possible to create the correction matrix separately for each method.

The particle selection query (6610) is then used to extract selected particle data (6630) from the database (6620). The calculate correction matrix step (6640) then includes the actual calculation of the new correction matrix. A grid size (6641) is needed for the calculation of a discrete matrix. It's size is determined by acceptable errors in calculating the correction. If the grid has only a few points large errors may occur even if interpolation is used between the points. A very large matrix will require more memory which might not be possible for example in an embedded system. Even a rather small matrix, such as 20×20, may be used. Particles for the calculation of each matrix value are selected in select particles for each grid point (6642). Remove outliers step (6643) is used in alternate embodiments. The median value (6644) of the set of particles for each point of the grid is selected as the corresponding value defining the correction factor for that point of the matrix.

The resulting new matrix and the old matrix (6650) extracted from the database (6620) are then compared (6660). Average values and the sum of average absolute differences of the points from the matrix mean are compared (Mat 1). These comparison results are then evaluated against limits; if the results are acceptable the new matrix is injected in to the database (6620) for use. If the results violate the limits an exception (6680) is raised, either in software or directly on the physical device in the form of a warning light or sound.

In an alternate embodiment the steps taken in the preferred embodiment are repeated except where a median was used to calculate the values of the correction matrix. In the alternate embodiment it is possible to use any method that is robust against outliers as step 6644, such as a robust version of LOESS or remove outliers separately (6643) and use any suitable statistic to calculate the matrix in 6644.

In another embodiment of the invention the correction information is retained in the form of a function, which may be continuous or piecewise continuous, and the correction value for each location is calculated only when needed.

In an alternate embodiment the database (6620) may hold a plurality of matrices for the evaluation of correction matrix change history such as the change in the speed of change of the matrix.

### EXAMPLES

The invention is illustrated by examples as follows, however, the applications where this invention provides advantages are not limited to these examples.

### Example 1

### Define an n by m Matrix

$${M}_{ij} = \{ \begin{matrix}{I}_{00} & ⋯ & {I}_{i 0} \\ ⋮ & ⋱ & ⋮ \\ {I}_{0 j} & ⋯ & {I}_{ij}\end{matrix} \}$$

where i=0, . . . , n−1 and j=0, . . . , m−1.

Further for a rectangular measurement area where X_{A}<x<X_{B}Λ Y_{A}<y<Y_{B} define

$${x}_{i} = i \frac{{X}_{B} - {X}_{A}}{n} + {X}_{A}$$

$$and$$

$${y}_{j} = j \frac{{Y}_{B} - {Y}_{A}}{m} + {Y}_{A}$$

Let particle measurement ξ be a list of brightness and location information Jₖ, xₖ, yₖ where k=1, . . . , p and denote the median value of a group of observations Jₖ with \tilde{J}ₖ then

Iᵢⱼ=\tilde{J}ₕ
where h is the subgroup of k that fulfills the selection condition σ_{φ} i.e. Jₕ=σ_{φ}(ξₖ).

A condition φₛ where s (s≤k) closest neighbours are selected can be implemented by selecting all ξₖ where

rₖᵢⱼ=\sqrt{(xₖ−xᵢ)²+(yₖ−yᵢ)²}<rₛ₊₁

where rₖᵢⱼ is the distance of the measured particles from the point xᵢ, yⱼ and rₛ is the distance of s^{th} closest particle from the point of interest xᵢ, yⱼ.

In a similar manner a selection condition φᵣ can be defined by selecting all ξₖ where

rₖᵢⱼ=\sqrt{(xₖ−xᵢ)²+(yₖ−yᵢ)²}<r

where r is a distance from the point of interest xᵢ, yⱼ and

0<r<\sqrt{(X_{B}−X_{A})²+(Y_{B}−Y_{A})²}.

Here s and r function as smoothing parameters and are selected based on the particular qualities of the system in question.

Extrapolation of matrix Mᵢⱼ to positions that fall between the defined points can be done using any of the several well-known methods such as nearest neighbour, bi-linear or bi-cubic.

### Example 2

### Calculation of Correction Matrix from Particle Data

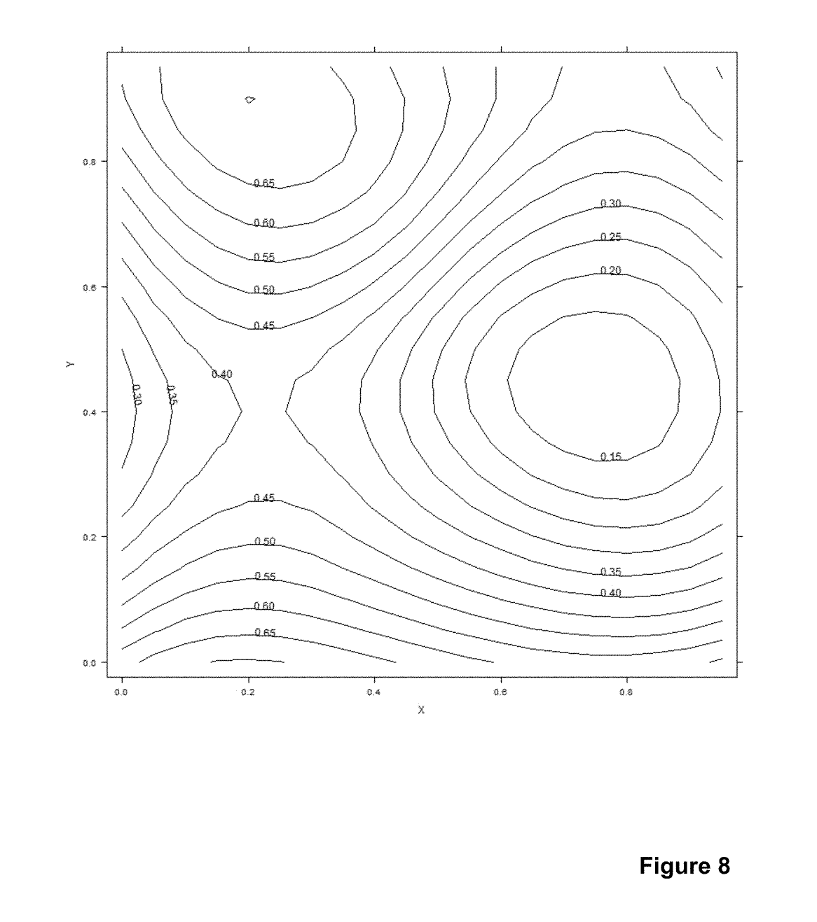

Referring to FIG. 6600, a particle selection query was made to the database to retrieve particle values with the following parameters: datetime between 2013-05-04 15:31:50 EEST and 2013-07-13 09:11:06 EEST, measurement type an actual measurement (i.e. not a control), errorstatus no errors, signal between 0.08 and 2 and the number of particles 1500. This resulted in a particle set shown in the histogram of FIG. 7. The signal values have been normalized with an arbitrary constant.

Selecting the closest sixty particles to point x1=0.55 and y1=0.40 results in a median of 0.17 and similarly selecting for location x2=0.35 and y2=0.85 gives a median of 0.69.

When values for the whole grid are calculated the result can be illustrated as a contour plot. In FIG. 8 to compare the median method with a more computationally intensive alternate, the contour plot was calculated directly from the particle data using the appropriate command from the R statistical computing software. There is a reasonable agreement with the values given above.

### Example 3

### Change of Correction Matrix with Time

Using the formula

$$S = {\sum }_{x , y} \vert z ( x , y ) \text{/} \text{<} z \text{>} - 1 \vert$$

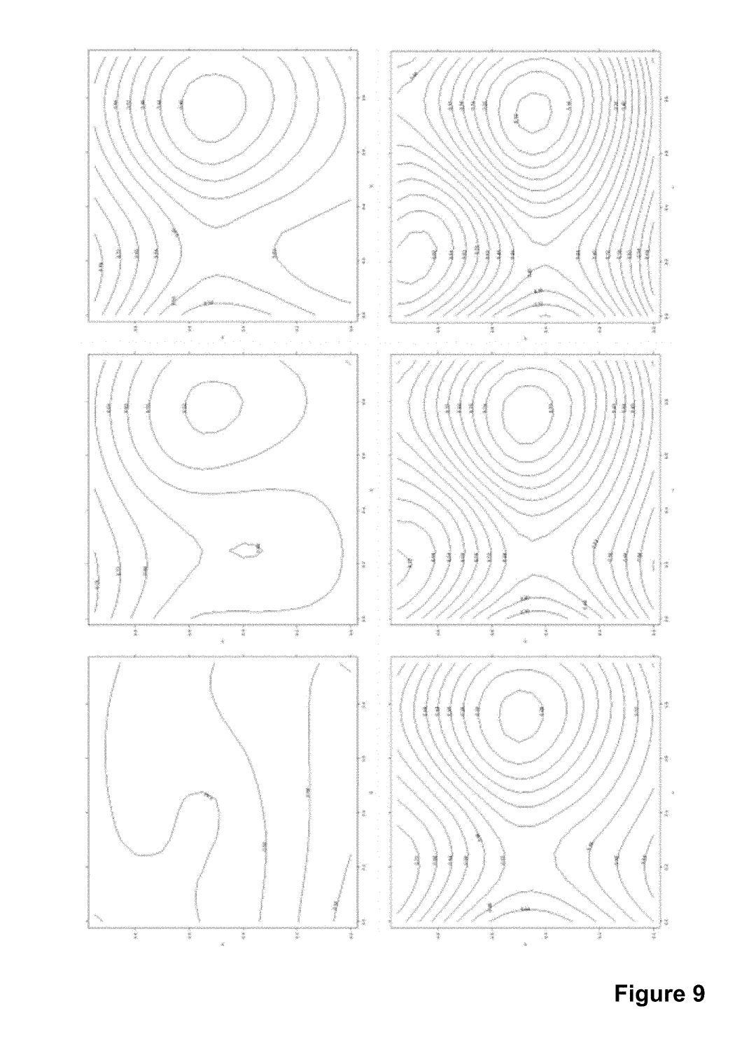

the deviance of the correction surface from an optimal one can be characterized, for this case of example 1 the value of S=120. FIG. 9 illustrates how the correction surface changes over time, right bottom corresponds to FIG. 8. The corresponding S values are: 23, 32, 53, 78, 101 and 122. If a limit of safe operation for the device was set to S=110, device operation would have been stopped when the last correction matrix was calculated (6670). After measuring for example the first four values it would also have been possible to forecast when approximately the limit will be exceeded as the rate of change of S is clearly visible and steady.

1. A separation free bioanalytical assay method for qualitatively and/or quantitatively determining an analyte (4) in a sample of a biological fluid or suspension, said method comprising the steps of:
a) contacting a bioaffinity solid phase comprising microparticles (1) to which a primary reagent (2) biospecific to said analyte (4) is bound simultaneously with said sample and a secondary reagent (3) biospecific to said analyte (4) labelled with a fluorescent label in a reaction volume, thereby initiating a reaction,
b) scanning a two-photon excitation focal volume within said reaction volume using a beam deflecting scanner and a two-photon exciting volume created by a focused laser beam which optically moves the microparticles (1),
c) momentarily interrupting scanning or reducing scanning speed of said two-photon excitation focal volume when said two-photon exciting volume approaches a microparticle (1) randomly located in the reaction volume,
d) applying optical force to said microparticle (1) such that it moves into and in the two-photon exciting volume created by said laser beam, and
e) detecting two-photon excited fluorescence emission photon counts from said microparticle (1);
characterized in that said method further comprises:
f) recording focus positions and corresponding two-photon excited fluorescence emission photon counts of a plurality of said microparticles (1) of a device;
g) calculating a correction matrix for said device by employing said recorded focus positions and said corresponding two-photon excited fluorescence emission photon counts, and
h) correcting two-photon excited fluorescence emission photon counts from said microparticles (1) of said device by employing said correction matrix obtained for said device by employing said recorded focus positions and said corresponding two-photon excited fluorescence emission counts.

2. The method of claim 1, characterized in that the correction matrix is recalculated continuously, or at pre-set intervals or time points, by employing the recorded focus positions and the corresponding two-photon excited fluorescence emission photon counts within a defined preceding time period.

3. The method of claim 2, characterized in that the preceding time period is chosen so that recorded focus positions and corresponding two-photon excited fluorescence emission photon counts of a minimum number of microparticles (1) are employed when calculating the correction matrix for the device.

4. The method of claim 1, characterized in that the correction matrix is calculated by employing the recorded focus positions and the corresponding two-photon excited fluorescence emission photon counts from microparticles (1) of at least one negative control sample, i.e. a sample or samples not comprising the analyte (4).

5. The method of claim 1, characterized in that the correction matrix is calculated by employing recorded focus positions and corresponding two-photon excited fluorescence emission photon counts of clinical sample measurements and employing only particles with two-photon excited fluorescence emission photon counts within a predetermined margin of the cut-off value for a positive result for an analyte (4).

6. The method of claim 1, characterized in that the two-photon excited fluorescence emission photon counts from individual microparticles (1) are normalized for the median of the fluorescence emission photon counts obtained during the measurement of a single well (20).

7. The method of claim 1, characterized in that the correction matrix is approximated by calculating an n by m matrix of correction factors where for each position of the correction matrix an approximate correction value is calculated from the two-photon excited fluorescence emission photon counts from said microparticles (1) that were detected within a set radius from said position.

8. The method of claim 7, characterized in that the approximate correction value is the median of the two-photon excited fluorescence emission photon counts.

9. The method of claim 1, characterized in that changes in the correction matrix are applied to determine changes in the health of the device, i.e. in device health, and/or need for maintenance of the device.

10. The method of claim 9, characterized in that the device is withdrawn from use until maintenance if the correction matrix changes beyond a set limit.

11. The method of claim 9, characterized in that the device is withdrawn from use until maintenance if the speed of change of the correction matrix exceeds a set limit.
